(19) United States
(12) Patent Application Publication
(10) Pub. No.: US 2020/0085881 A1
(43) Pub. Date: Mar. 19, 2020

(54) TISSUE HEALING AGENT

(71) Applicants: OSAKA AIR MACHINE SERVICE, LTD., Higashiosaka-shi, Osaka (JP); Regene Pharm Co., Ltd., Higashiosaka-shi, Osaka (JP)

(72) Inventors: Akifumi MATSUYAMA, Toyoake-shi (JP); Hanayuki OKURA, Higashiosaka-shi (JP)

(73) Assignees: OSAKA AIR MACHINE SERVICE, LTD., Higashiosaka-shi, Osaka (JP); Regene Pharm Co., Ltd., Higashiosaka-shi, Osaka (JP)

(21) Appl. No.: 16/469,710

(22) PCT Filed: Dec. 15, 2017
(86) PCT No.: PCT/JP2017/045037

§ 371 Date: Jun. 14, 2019

(30) Foreign Application Priority Data
Dec. 16, 2016 (JP) 2016-244724

(51) Int. Cl. A61K 35/51; A61K 35/28; A61K 38/21; A61K 38/20; A61K 38/19; A61K 38/18; A61K 35/50; A61P 9/00; A61P 1/16

(52) U.S. Cl. CPC A61K 35/51; A61K 35/28; A61K 38/215; A61K 38/217; A61K 38/2006; A61K 38/20; A61P 1/16; A61K 38/1841; A61K 38/1808; A61K 38/1825; A61K 35/50; A61P 9/00; A61K 38/191

(57) ABSTRACT

The present invention provides a pharmaceutical composition for healing tissue, said pharmaceutical composition containing: adherent cells originating from mesenchymal tissue that has been treated with a physiologically active polypeptide or an LPS; and a pharmaceutically acceptable carrier.

### TECHNICAL FIELD

The present invention relates to a pharmaceutical composition for tissue healing, and a method for producing the same. In particular, the present invention relates to a tissue healing agent containing drug-treated adherent cells derived from mesenchymal tissue, and a method for producing the same.

### BACKGROUND ART

Mesenchymal tissue-derived cells have been shown to be useful for tissue healing. Among them, mesenchymal stem cells (MSCs) are being actively studied for their clinical application in regenerative medicine. For example, tissue is considered as a source of stem cells (ASCs) (Non-Patent Document 1), and ASCs are known to have therapeutic effects in various areas (Non-Patent Document 2). In addition, adipose tissue-derived multilineage progenitor cells (ADMPCs) have also been shown to be effective for treatment of liver diseases (Patent Document 1).

Thus, mesenchymal tissue-derived cells have been shown to be useful in regenerative medicine involving tissue healing. Accordingly, further improvement of their healing ability is desired.

### PRIOR ART DOCUMENTS

### Patent Document

Patent Document 1: WO 2008/153179

### Non-Patent Documents

Non-Patent Document 1: Zuk P A, Zhu M, Ashjian P, et al. Human adipose tissue is a source of multipotent stem cells. MoI Biol Cell 2002; 13: 4279-4295.

Non-Patent Document 2: Japanese Journal of Transfusion and Cell Therapy, Vol. 59, No. 3: 450-456, 2013

### SUMMARY OF THE INVENTION

### Problems to be Solved by the Invention

It is an object to further improve the tissue healing ability of mesenchymal tissue-derived cells.

### Solutions to the Problems

As a result of intensive studies to solve the above problems, the present inventors have found that adherent cells derived from mesenchymal tissue treated with a physiologically active polypeptide or LPS (lipopolysaccharide) have extremely high tissue healing ability, and the present invention has been completed.

That is, the present invention provides the followings.

(1) A pharmaceutical composition for tissue healing, including adherent cells derived from mesenchymal tissue treated with a physiologically active polypeptide or LPS, and a pharmaceutically acceptable carrier.

(2) The pharmaceutical composition according to (1), wherein the physiologically active polypeptide is one or more polypeptides selected from the group consisting of inflammatory cytokine, inflammatory cytokine-inducing polypeptide, growth factor, chemokine, hormone and interferon.

(3) The pharmaceutical composition according to (1) or (2), wherein the physiologically active polypeptide is one or more polypeptides selected from the group consisting of interferon-β (IFN-β), interferon gamma (IFNγ), interleukin-1 alpha (IL-1α), interleukin-1 beta (IL-1β), interleukin-17A (IL-17A), tumor necrosis factor alpha (TNFα), tumor necrosis factor beta (TNFβ), type I interferon (INF-I), transforming growth factor β (TGFβ), epidermal growth factor (EGF) and fibroblast growth factor (FGF).

(4) The pharmaceutical composition according to any one of (1) to (3), wherein the adherent cells derived from mesenchymal tissue are mesenchymal tissue-derived stem cells (MSCs), adipose tissue-derived multilineage progenitor cells (ADMPCs), umbilical cord tissue-derived cells, placenta tissue-derived cells, or bone marrow tissue or synovium tissue-derived cells.

(5) The pharmaceutical composition according to any one of (1) to (4), wherein the tissue healing is tissue protection, repair of tissue/cell injury, promotion of proliferation of cells constituting a tissue, suppression of tissue inflammation or reconstruction of tissue form.

(6) The pharmaceutical composition according to any one of (1) to (5), wherein the tissue healing is tissue healing in chronic phase disease.

(7) The pharmaceutical composition according to any one of (1) to (6), wherein the tissue is liver tissue or cardiac tissue.

(8) A method for producing a pharmaceutical composition for tissue healing, including the steps of:

(a) treating adherent cells derived from mesenchymal tissue with a physiologically active polypeptide or LPS, and

(b) mixing the cells treated in step (a) with a pharmaceutically acceptable carrier.

(9) The method according to claim 1, wherein the physiologically active polypeptide is one or more polypeptides selected from the group consisting of inflammatory cytokine, inflammatory cytokine-inducing polypeptide, growth factor, chemokine, hormone and interferon.

(10) The method according to (8) or (9), wherein the physiologically active polypeptide is one or more polypeptides selected from the group consisting of interferon-β (IFN-β), interferon gamma (IFNγ), interleukin-1 alpha (IL-1α), interleukin-1 beta (IL-1β), interleukin-17A (IL-17A), tumor necrosis factor alpha (TNFα), tumor necrosis factor beta (TNFβ), type I interferon (INF-I), transforming growth factor β epidermal growth factor (EGF) and fibroblast growth factor (FGF).

(11) The method according to any one of (8) to (10), wherein the adherent cells derived from mesenchymal tissue are mesenchymal tissue-derived stem cells (MSCs), adipose tissue-derived multilineage progenitor cells (ADMPCs), umbilical cord tissue-derived cells, placenta tissue-derived cells, or bone marrow tissue or synovium tissue-derived cells.

(12) The method according to any one of (8) to (11), wherein the tissue healing is tissue protection, repair of tissue/cell injury, promotion of proliferation of cells constituting a tissue, suppression of tissue inflammation or reconstruction of tissue form.

(13) The method according to any one of (8) to (12), wherein the tissue healing is tissue healing in chronic phase disease.

(14) The method according to any one of (8) to (13), wherein the tissue is liver tissue or cardiac tissue.

### EFFECTS OF THE INVENTION

According to the present invention, a pharmaceutical composition having an extremely high tissue healing ability can be obtained. The pharmaceutical composition of the present invention is useful for tissue healing in chronic phase-tissue injury and the like.

### BRIEF DESCRIPTION OF DRAWINGS

FIG. 1 is a graph comparing the produced amount of adiponectin in adipose tissue-derived multilineage progenitor cells (ADMPCs) treated with IL-1β (left) and the produced amount of adiponectin in ADMPCs not treated with IL-1β (right). The vertical axis represents the amount of adiponectin produced.

FIG. 2 is a graph comparing the produced amount of hepatocyte growth factor (HGF) in ADMPCs treated with IL-1β (left) and the produced amount of HGF in ADMPCs not treated with IL-1β (right). The vertical axis represents the amount of HGF produced.

FIG. 3 is an image of a Sirius red-stained tissue section showing decrease in intrahepatic fibers by ADMPCs treated with IL-1β in non-alcoholic steatohepatitis (NASH) model mice. The result of a carrier administered group is in the left, the result of an administrated group with ADMPCs not treated with IL-1β is in the middle, and the result of an administered group with ADMPCs treated with IL-1β is in the right. The magnification is 50 times.

FIG. 4 is an image of a HE-stained tissue section showing reduction in liver tissue injury by ADMPCs treated with IL-1β in non-alcoholic steatohepatitis (NASH) model mice. The result of a carrier administered group is in the left, the result of an administrated group with ADMPCs not treated with IL-1β is in the middle, and the result of an administered group with ADMPCs treated with IL-1β is in the right. The magnification on the top panels is 50 times, and the magnification on the bottom panels is 200 times.

FIG. 5 is a graph comparing reduction in liver tissue injury by ADMPCs treated with IL-1β and ADMPCs not treated with IL-1β in non-alcoholic steatohepatitis (NASH) model mice using NAFLD Activity Score. The p value is according to Mann-Whitney's U test.

FIG. 6 is a graph illustrating improvement in left ventricular ejection fraction by ADMPCs treated with IL-1β and ADMPCs not treated with IL-1β in severe myocardial infarction model animals (pigs). The vertical axis (AEF %) represents change (%) in left ventricular ejection fraction before and after administration of cells. The white bar represents a control group to which cells are not administered, the hatched bar represents a group to which ADMPCs not treated with IL-1β are administered, and the black bar represents a group to which ADMPCs treated with IL-1β are administered.

### MODE FOR CARRYING OUT THE INVENTION

In one aspect, the present invention provides pharmaceutical composition for tissue healing, including adherent cells derived from mesenchymal tissue treated with a physiologically active polypeptide or LPS, and a pharmaceutically acceptable carrier. Here, the physiologically active polypeptide is a polypeptide that acts on a certain physiological regulatory function of the living body. Polypeptide refers to a substance in which two or more amino acid residues are linked to each other via a peptide bond. Various types of LPS are known, and any LPS may be used.

The physiologically active polypeptide used in the present invention also includes its variants. The variant of the physiologically active polypeptide is one having an activity capable of, when acted on mesenchymal tissue-derived adherent cells, providing mesenchymal tissue-derived adherent cells that can be used for tissue healing of the present invention.

The variant refers to a polypeptide in which the amino acid residue constituting the polypeptide has been substituted, deleted or added, with respect to the original peptide. The number of amino acid residues to be substituted, deleted or added is not particularly limited. For example, one to several amino acid residues may be substituted, deleted or added. For example, the variant polypeptide may have an amino acid sequence identity of 80% or more, preferably 90% or more, for example 95% or more, 97% or more, or 99% or more, with respect to the original polypeptide. Furthermore, the variant of the physiologically active polypeptide may be one in which the amino acid residue constituting the polypeptide is modified. The modification may be with any type of label. The modification may be chemical modification such as methylation, halogenation or glycosylation, or labeling such as fluorescence labeling or radioactive labeling. The variant of the physiologically active polypeptide may be one in which some amino acid residues are linked to each other via a bond other than a peptide bond.

The physiologically active polypeptide used in the present invention may be any polypeptide. Suitable physiologically active polypeptides used in the present invention are preferably cytokine, in particular one or more polypeptides selected from the group consisting of inflammatory cytokine, inflammatory cytokine-inducing polypeptide, growth factor, hormone and interferon. The inflammatory cytokine is a cytokine involved in pathogenesis of inflammation. The inflammatory cytokine-inducing polypeptide is a polypeptide having an effect of increasing the amount of inflammatory cytokine or enhancing the activity thereof. The growth factor is a polypeptide that promotes the growth or differentiation of specific cells in vivo. The chemokine is a basic protein that exhibits the action via a G protein coupled receptor and is a group of cytokines. The hormone is a substance that is produced in vivo, transported via body fluids, and affects the activity of specific cells, tissue or organ. The interferon is a group cytokines produced in response to entry of foreign substances such as virus, pathogen or tumor cells in vivo. Various inflammatory cytokines, inflammatory cytokine-inducing polypeptides, growth factors and interferons are publicly known and any of them may be used.

The cytokines include, but are not limited to, IL-1α, IL-1β, IL-2 to IL-35, OSM (Oncostatin M), LIF, CNTF, CT-1, TNF-α, TNF-β, BAFF, FasL, RANKL and TRAIL. The inflammatory cytokines include, but are not limited to, IL-1α, IL-1β, IL-6, IL-8, IL-12, IL-18 and TNFα.

The inflammatory cytokine -inducing polypeptides include, but are not limited to, IL-17A.

The growth factors include, but are not limited to, activin A, ANGPTL5, BAFF, BD-2, BD-3, BNDF, BMP-1 to 7, DKK1, EGF, EG-VEGF, FGF-1 to 21, G-CSF, GM-CSF, HGF, IGF-1, IGF-2, platelet-derived growth factor (PDGF)-AA, PDGF-AB, PDGF-BB, R-spondin-1 to 3, SCF, galectin-1 to 3, GDF-11, GDNF, pleiotrophin, TGF-α, TGF-β, TPO (thrombopoietin), TSLP, vascular endothelial growth factor (VEGF) and ciliary neurotrophic factor (CNTF).

The chemokines include, but are not limited to, CCL1 to CCL28 and CXCL1 to CXCL10.

The hormones include, but are not limited to, Calcitonin, Parathormone, Glucagon, Erythropoietin, Leptin, ANP, BNP, CNP, Oxytocin, Vasopressin, TRH (thyrotropin releasing hormone), TSH (thyroid stimulating hormone), CRH (corticotropin releasing hormone), ACTH (adrenocorticotropin hormone), GRH (gonadotropin releasing hormone), FSH (follicle stimulating hormone), LH (luteinizing hormone), SOM (somatostatin), GRH (growth hormone releasing hormone), GH (growth hormone), PRH (prolactin releasing hormone), PIH (prolactin inhibiting hormone) and Prolactin.

The interferons include, but are not limited to, IFN-α, IFN-β, IFN-γ and IFN-I.

Suitable physiologically active peptides used in the present invention are inflammatory cytokine, inflammatory cytokine-inducing polypeptide, growth factor and interferon. Among them, preferable examples include, but are not limited to, IFN-β, IFN-γ, IL-1α, IL-1β, IL-17A, TNFα, INF-β, INF-I, TGFβ, EGF and FGF.

The tissue healing refers to restoring a tissue to a normal state or bringing a tissue closer into a normal state, including tissue protection, repair of tissue/cell injury, promotion of proliferation of cells constituting a tissue, suppression of tissue inflammation, wound healing and reconstruction of the tissue form. Because the cells in the pharmaceutical composition of the present invention are useful for tissue protection, promotion of proliferation of cells constituting a tissue, etc., the pharmaceutical composition of the present invention is preferably used for tissue healing in chronic phase disease.

Tissues to be healed by the pharmaceutical composition of the present invention are any tissue of animal and are not particularly limited. Examples of the tissues include, but are not limited to, liver, pancreas, kidney, muscle, bone, cartilage, bone marrow, stomach, intestine, blood, nerve, skin, mucous membrane, heart and hair. Suitable tissues to be healed by the pharmaceutical composition of the present invention are liver, nerve, skin, mucous membrane and heart. Therefore, the pharmaceutical composition of the present invention is preferably used for treatment of, for example, liver cirrhosis, hepatitis and NASH (nonalcoholic steatohepatitis), and is also effective for chronic phase disease.

The cells that are an active ingredient of the pharmaceutical composition of the present invention are adherent cells derived from mesenchymal tissue treated with a physiologically active polypeptide or LPS.

The pharmaceutical composition of the present invention may be administered to a subject in the same species as or different species from the animal species from which the active ingredient cells are derived. For example, the pharmaceutical composition of the present invention including adherent cells derived from human-derived mesenchymal tissue treated with an inflammatory cytokine-inducing agent may be administered to a human subject. The cells in the pharmaceutical composition of the present invention may be from the same subject as the subject to be administered, or may be from a different subject from the subject to be administered.

Any mesenchymal tissue-derived adherent cells may be used in the present invention. The mesenchymal tissue-derived adherent cells may be commercially available ones, for example, distributed ones from organizations such as American Type Culture Collection (ATCC) (US) and NITE (Japan). Alternatively, mesenchymal tissue-derived adherent cells may be obtained from mesenchymal tissue. Means and methods for preparing mesenchymal tissue-derived adherent cells from mesenchymal tissue are publicly known.

Examples of suitable mesenchymal tissue-derived adherent cells include mesenchymal tissue-derived stem cells (MSCs) such as adipose tissue-derived stem cells (ASCs), adipose tissue-derived multilineage progenitor cells (ADMPCs), Muse cells, cells derived from bone marrow tissue, umbilical cord tissue, amniotic tissue, cartilage tissue, periosteum tissue, synovium tissue, skeletal muscle tissue and placenta tissue, stem cells and stromal cells, and menstrual blood cells.

When the cells are obtained from mesenchymal tissue, they may be isolated from any mesenchymal tissue. Examples of mesenchymal tissue include, but are not limited to, adipose tissue, bone marrow tissue, umbilical cord tissue, amniotic tissue, cartilage tissue, periosteum tissue, synovium tissue, skeletal muscle tissue, placenta tissue and menstrual blood. Suitable mesenchymal tissues include adipose tissue, bone marrow tissue and umbilical cord tissue. In particular, adipose tissue is preferable because it is contained in a large amount in the body and many cells can be extracted.

Adherent cells can be obtained by extracting mesenchymal tissue from the body, placing and culturing the tissue in a culture vessel, and selectively acquiring cells adhering to the vessel. Mesenchymal tissue can be extracted using publicly known means and methods such as excision and aspiration. The extracted mesenchymal tissue may be cultured as it is, or if necessary, the extracted mesenchymal tissue may be minced or broken, followed by removing of red blood cells to culture the obtained cell population. These treating methods and means, and cell culturing means and methods are publicly known, and can be appropriately selected. Mesenchymal tissue-derived adherent cells may be obtained, for example, by treating the cells attached to the culture vessel with an enzyme such as trypsin.

Treatment of mesenchymal tissue-derived adherent cells with a physiologically active polypeptide or LPS may be carried out by contacting the cells with cytokine in a publicly known manner. Typically, this treatment may be carried out by culturing mesenchymal tissue-derived adherent cells for a certain period of time in a medium containing an appropriate concentration of physiologically active polypeptide or LPS. Usually, mesenchymal tissue-derived adherent cells are cultured in several nanograms/ml to several hundred nanograms/ml of inflammatory cytokine or a medium to which inflammatory cytokine has been added. The medium for use in culture may be a publicly known one. The culturing time and culturing temperature may also be appropriately selected. If necessary, mesenchymal tissue-derived adherent cells may be cultured to increase the number of cells, before treatment with a physiologically active polypeptide or LPS. A desired subpopulation may be obtained from a population of mesenchymal tissue-derived adherent cells, and if necessary, the subpopulation may be cultured to increase the number of cells, before treatment with physiologically active polypeptide or LPS.

The number of types of physiologically active polypeptide or LPS for use in treatment of mesenchymal tissue-derived adherent cells may be one or two or more.

Mesenchymal tissue-derived adherent cells treated with a physiologically active polypeptide or LPS increases expression and production of one or more factors that contribute to tissue repair (such as polypeptide, growth factor and/or enzyme involved in tissue healing), or expresses and produces the same. Such factors include, but are not limited to, adiponectin, HGF, CSF2 (GM-CSF), CSF3 (G-CSF), LIF, MMP family factors, FGF family factors, ADAM factors, angiopcietin-like protein family factors, pleiotrophin, R-spondin family factors and VEGF family factors. CSF2 or CSF3 contributes not only to activation of hematopoietic stem cells but also to stem cell proliferation and/or angiogenesis in many tissues or organs including brain, heart, lung and liver, thereby contributing to tissue repair. Accordingly, cells that express and produce these factors at higher level are preferred. The increase in expression or production of the above factor may be, for example, 10 times or more, preferably 30 times or more, more preferably 50 times or more, still more preferably 100 times or more, compared to that before treatment.

The pharmaceutical composition of the present invention can be produced by mixing mesenchymal tissue-derived adherent cells treated with a physiologically active polypeptide or LPS as described above with a pharmaceutically acceptable carrier. A variety of pharmaceutically acceptable carriers are publicly known and may be appropriately selected for use. For example, when the pharmaceutical composition of the present invention is used as an injection, the cells may be suspended in a carrier such as purified water, saline or phosphate buffered saline.

The dosage form of the pharmaceutical composition of the present invention is not particularly limited, but may be a solution, semisolid or solid. The administration method of the pharmaceutical composition of the present invention as also not limited, but may include local injection, intravenous injection or infusion, application to an affected area, administration to an affected area via a catheter, or direct transplantation to tissues such as liver by a surgical procedure. The pharmaceutical composition of the present invention may be transplanted in the form of cell sheet, cell mass, layered cell sheet, etc.

The administration route and dose of the pharmaceutical composition of the present invention may be appropriately determined in consideration of the type and site of the tissue to be healed, the degree of disease, the condition of the subject and the like.

The pharmaceutical composition of the present invention may contain cells other than adherent cells derived from mesenchymal tissue treated with a physiologically active polypeptide or LPS.

In a further aspect, the present invention provides use of adherent cells derived from mesenchymal tissue treated with a physiologically active polypeptide or LPS in producing a medicament for tissue healing.

In a further aspect, the present invention provides use of adherent cells derived from mesenchymal tissue treated with a physiologically active polypeptide or LPS for tissue healing.

In a further aspect, the present invention provides a method for tissue healing in a subject in need of tissue healing, including administering to the subject adherent cells derived from mesenchymal tissue treated with a physiologically active polypeptide or LPS.

In yet another aspect, the present invention provides a method for producing a pharmaceutical composition for tissue healing, including the steps of:

(a) treating adherent cells derived from mesenchymal tissue with a physiologically active polypeptide or LPS, and

(b) mixing the cells treated in step (a) with a pharmaceutically acceptable carrier.

In yet another aspect, the present invention provides a method for producing cells for tissue healing, including treating adherent cells derived from mesenchymal tissue with a physiologically active polypeptide or LPS.

Hereinafter, more detailed and specific description is made of the present invention with reference to Examples, but the Examples are not intended to limit the present invention.

### EXAMPLE 1

### Example 1. Effect of Treating ADMPCs with IL-1β

(1) Method of Experiment

(i) Collection of Adipose Tissue from Human Subject

From six women from which informed consent was obtained, extra adipose tissue to be discarded was received during liposuction surgery. The protocol conformed to the Kobe University Graduate School of Medicine Review Boards for Human Research.

(ii) Isolation and Culture of ADMPCs

The adipose tissue was minced and then digested in Hanks' buffered saline solution (HESS) containing 0.008% Liberase (Roche Lifescience) with shaking in a water bath at 37° C. for 1 hour. The digested product was filtered through Cell Strainer (BD Bioscience), followed by centrifuging at 800×g for 10 minutes. The lymphocyte separation solution (d=1.077) (Nacalai tesque) was used to remove red blood cells by specific gravity method. Cells in the obtained cell population containing ADMPCs were seeded in DMEM containing 10% fetal bovine serum (Hyclone) to allow for attachment of the cells, followed by washing and treatment with EDTA to yield ADMPCs. Then, the ADMPCs in a medium (60% DMEM-low glucose, 40% MCDB201, 10 μg/mL EGF, 1 nM dexamethasone, 100 μM ascorbic acid and 5% FBS) were seeded on a human fibronectin-coated dish and subcultured 3 to 8 times to yield cultured ADMPCs.

(iii) IL-1β Treatment

IL-1β was added to a medium (60% DMEM-low glucose, 40% MODE 201, 10 μg/mL EGF, 1 nM dexamethasone, 100 μM ascorbic acid and 5% FES) to a concentration of 10 ng/ml. The cultured ADMPCs obtained in (ii) above were cultured in the IL-1β-containing medium for 72 hours to measure adiponectin and hepatocyte growth factor (HGF) produced in the medium. The measurement of adiponectin was performed using the ELISA kit of abeam (Catalog No. ab99968). The measurement of HGF was performed using the ELISA kit of R & D System (Catalog No. DHG00). For a control, ADMPCs were cultured in the above medium except that IL-1β was not added.

(2) Results of Experiment

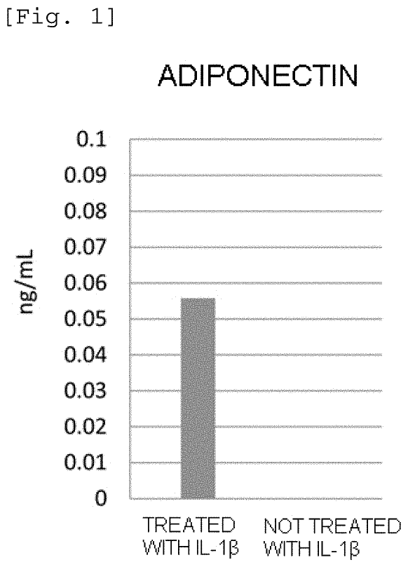

The measurement results of amount of adiponectin produced are shown in FIG. 1. Production of adiponectin from ADMPCs not treated with IL-1β was not found, but it was confirmed that adiponectin was produced from ADMPCs treated with IL-1β.

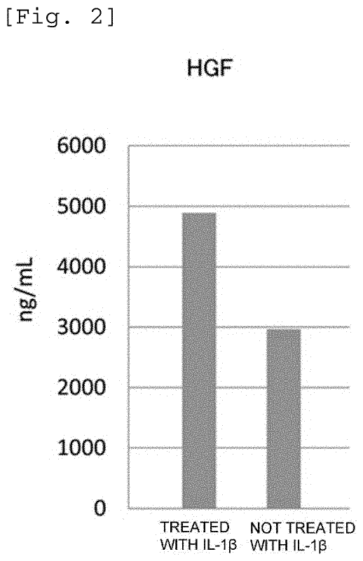

The measurement results of amount of HGF produced are shown in FIG. 2. The amount of HGF produced from ADMPCs treated with IL-1β was increased by about 1.7 times as compared to the amount of HGF produced from ADMPCs not treated with IL-1β.

### Example 2

Example 2. In Vivo Tissue Healing Effect of ADMPCs Treated with IL-1β—Alleviation of Liver Tissue Injury

(1) Method of Experiment

Collection of adipose tissue from a human subject, isolation and culture of ADMPCs, and IL-1β treatment were performed in the same manner as in Example 1, except that the concentration of IL-1β in the medium was 5 ng/ml.

ADMPCs treated with IL-1β were suspended in a carrier to a concentration of 1.2×10⁵ cells/ml. This was administered to NASH model mice (STAN (registered trademark) mice) to examine healing of the liver tissue. The animals were divided into 3 groups: administrated group with ADMPCs treated with IL-1β (n=9), administrated group with ADMPCs not treated with IL-1β (n=9), and carrier administered group (n=10)). At the beginning of the study, animals in each group were intradermally administered with streptozotocin, fed with a normal diet, fed with a high-fat diet from week 4 to week 9, and euthanized at week 9. Administration of ADMPCs (3×10⁵ cells/kg) and carrier was performed once at week 6. The liver tissue sections obtained were subjected to Sirius red staining and hematoxylin-eosin (HE) staining.

(2) Results of Experiment

(i) Sirius Red Staining of Liver Tissue Sections

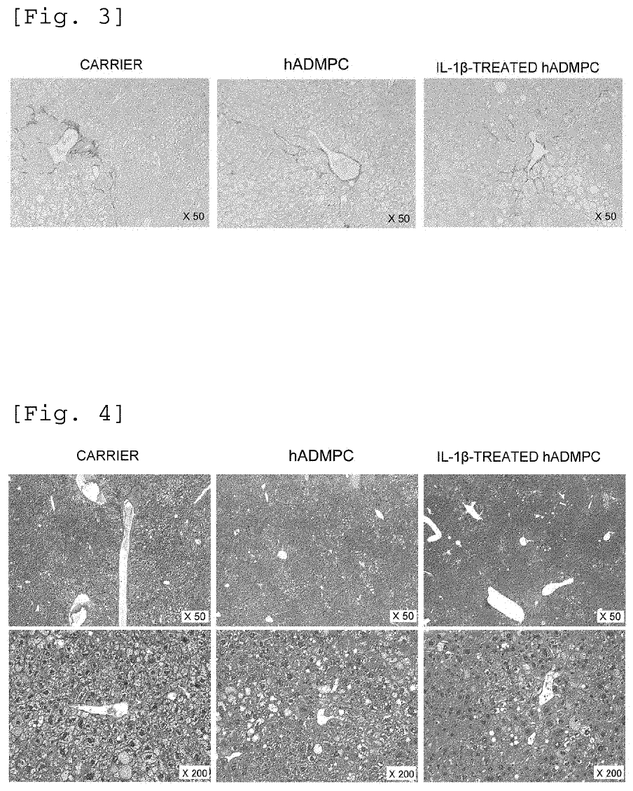

The results of Sirius red staining of liver tissue sections from mice obtained at week 9 of the study are shown in FIG. 3. In the livers from mice administered with ADMPCs treated with IL-1β, it was confirmed that deposition of intrahepatic fibers stained with Sirius red was reduced, compared to the livers from carrier administered mice and mice administered with ADMPCs not treated with IL-1β.

(ii) HE Staining of Liver Tissue Sections

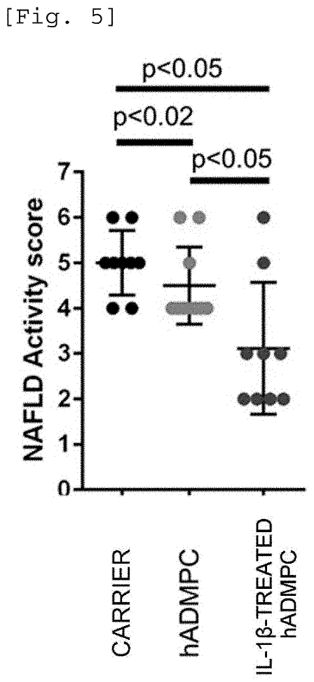

The results of HE staining of liver tissue sections from mice obtained at week 9 of the study are shown in FIG. 4. In the livers from mice administered with ADMPCs treated with IL-1β, it was confirmed that injury to liver tissue represented by vacuolation was reduced, compared to the livers from carrier administered mice and mice administered with ADMPCs not treated with IL-1β.

(iii) Evaluation of Liver Tissue Healing Effect by NAFLED Activity Score (E. M. Brunt et al. Hepatology. 2011 March; 53(3): 810-820)

The degree of liver tissue injury in mice obtained at week 9 of the study was evaluated according to the NAFLED activity score. The evaluation method of NAFLED activity score is shown in Table 1.

TABLE 1
EVALUATION METHOD OF NAFLD ACTIVITY SCORE

| ITEM | DEFINITION | SCORE |
|---|---|---|
| FATTY CHANGE | FATTY CHANGE AT LOW TO | |
| | MODERATE MAGNIFICATION | |
| | <5% | 0 |
| | 5-33% | 1 |
| | 33-66% | 2 |
| | >66% | 3 |
| INFLAMMATION OF | EVALUATION OF INFLAMMATION | |
| LIVER | FOCI | |
| PARENCHYMA | NONE | 0 |
| | LESS THAN 2 SITES AT 200 | 1 |
| | TIMES ENLARGEMENT | |
| | 2 TO 4 SITES AT 200 TIMES | 2 |
| | ENLARGEMENT | |
| | MORE THAN 5 SITES AT 200 | 3 |
| | TIMES ENLARGEMENT | |
| LIVER CELL | NONE | 0 |
| INJURY | 2 TO 3 BALLOONING CELLS | 1 |
| (BALLOONING) | 4 OR MORE BALLOONING CELLS | 2 |

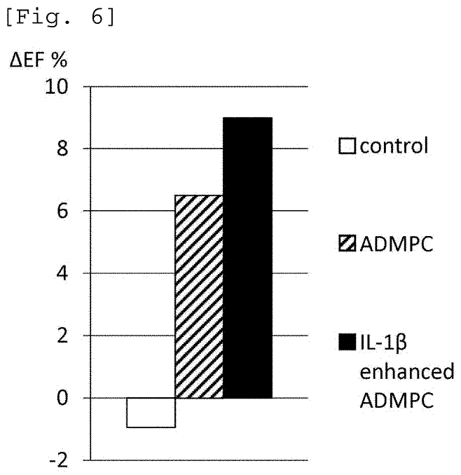

The results are shown in FIG. 5. Because the NAFLD activity score of the livers from mice administered with ADMPCs treated with IL-1β was significantly lower, compared to the livers from carrier administered mice and mice administered with ADMPCs not treated with IL-1β, it was confirmed that injury to liver tissue represented by vacuolation, inflammation and fatty change was greatly reduced.

From these results, ADMPCs treated with IL-1β are found to be effective in healing injured tissue, and be useful for tissue protection, repair of tissue/cell injury, suppression of tissue inflammation and promotion of proliferation of cells constituting a tissue. These tissue healings are considered to allow for reconstruction of the tissue form and wound healing. Moreover, because these effects were observed in mice in which liver injury was induced by streptozotocin and high-fat diet, it can be said that IL-1β-treated ADMPCs are effective for tissue healing in chronic phase disease.

### Example 3

Example 3. In Vivo Tissue Healing Effect of ADMPCs Treated with IL-1β—Improvement of Cardiac Function

### (1) Method of Experiment

A severe myocardial infarction model was created using 8 weeks old pigs by two-stage embolism/reperfusion method. Specifically, a 6F guide catheter was transcutaneously placed through the femoral artery on the opening of the left coronary artery, a guide wire was inserted through the catheter into the first diagonal artery (#9 in the AHA classification), and preconditioning was performed by conducting ballooning (obstruction reopening) with the aid of the guide. One week later, a guidewire was inserted into the left anterior descending coronary artery (#6 to #8 in the AHA classification), and ballooning (obstruction reopening) was performed at the left anterior descending coronary artery immediately below the bifurcation of the left circumflex coronary artery (#6 in the AMA classification) to produce a myocardial ischemic region. Four weeks after that (five weeks after the first obstruction reopening), individuals with a cardiac ejection fraction of 40% or less in cardiac ultrasonography were subjected to the study as a severe heart failure model.

Four weeks after the second embolism/reperfusion, the animals were divided into 3 groups: a control (cells were not administrated) group, group to which non-activated cells (ADMPCs) were administered at a concentration of 3×10⁵ cells/kg body weight through a catheter via the coronary artery, and group to which IL-1β activated cells (72 hours cultured) (IL-1β-activated ADMPCs) were administered in the same manner. Preparation of ADMPCs and IL-1β-activated ADMPCs was carried out in the same manner as in Example 1. Immediately before administration, cardiac MRI was performed 3 months after administration (Sigma EXCITE XI TwinSpeed 1.5T Ver. 11.1, GE Healthcare), using Cardiac Vx (GE Healthcare) as analysis software, to measure left ventricular end-diastolic and end-systolic volumes.

The Formula:

Left ventricular ejection fraction=100×(left ventricular end-diastolic volume−left ventricular end-systolic volume)/(left ventricular end-diastolic volume)

was used to calculate left ventricular ejection fraction (% EF) to represent the difference between the value 3 months after administration and the value immediately before administration as ΔEF (%) ((FIG. 6).

(2) Results of Experiment

As shown in FIG. 6, the left ventricular ejection fraction was decreased in the control group, whereas the left ventricular ejection fraction was improved in the two groups to which cells were administered, in partcular, when IL-1β-activated cells were administered, the left ventricular ejection fraction was markedly improved.

These results indicate that IL-1β-treated ADMPCs heal cardiac tissue injured by severe myocardial infarction and markedly improve the cardiac function.

### Example 4

### Example 4. Effect of Treating Adhesive Cells Derived from Various Mesenchymal Tissues with Various Physiologically Active Polypeptides

(I) Method of Experiment

As test cells, umbilical cord-derived mesenchymal stem cells (umbilical cord-derived MSCs), adipose tissue-derived stem cells (ADSCs), knee cartilage synovium-derived mesenchymal stem cells (synovium-derived MSCs), adipose tissue-derived multilineage progenitor cells (ADMPCs), placenta-derived mesenchymal stem cells (placenta-derived MSCs) and bone marrow-derived mesenchymal stem cells (bone marrow-derived MSCs) were used. Various cytokines, chemokines, growth factors and hormones were used as physiologically active polypeptides.

When ADMPCs were used as test cells, cytokines (IL-1α, IL-1β, IL3 to IL35, oncostatin M, LIF, CNTF, CT-1, TNFα, TNFβ, BAFF, FasL, RANKL, TRAIL, INF-α, IFN-β, IFN-γ), chemokines (CCL1 to CCL28, CXCL1 to CXCL10), growth factors (AvinA, ANGPLTS, BD-2, BD-3, BDNF, BMP-1 to BMP-7, DKK1, EGF, EG-VEGF, FGF-1 to FGF-21, G-CSF, HGF, IGF-1, IGF-2, PDGF-AA, PDGF-BB, R-spondin-1, R-spondin-2, R-sporadin-3, SCF, galectin 1, galectin 2, galectin 3, GDF-11, GDNF, pleiotrophin, TGFα, TGF3, TPO, TSLP, VEGF), and hormones (calcitonin, parathormone, glucagon, erythropoietin, leptin, ANP, BNP, CNP, oxytocin, vasopressin, TGH, TSH, CRH, ACTH, GRH, FSH, LH, SOM, GRH, GH, PRH, prolactin) were used as physiologically active polypeptides. When ADSCs, placenta-derived MSCs, synovium-derived MSCs, bone marrow-derived MSCs and umbilical cord-derived MSCs are used as test cells, IL-1α, IL-1β, TNFα, TNF, IFN-β, IFN-γ, FGF15 are used as physiologically active polypeptides. Hereinafter, the physiologically active polypeptide is referred to as “drug”. The test cells were subjected to medium replacement with a drug-containing medium (final concentration of 100 ng/mL) and drug-free medium (control), and further subcultured for 3 days (72 hours). After 72 hours of medium change, 600 μL of RLT Buffer was added for recovery and RNA extraction.

As to RLT Buffer samples, total RNA was extracted using RNeasy Mini Kit/QIAGEN, and the total RNA was prepared in concentration of 100 ng/μL. Then, labeled cRNA was synthesized from 150 ng of the total RNA per array. For the synthesized labeled cRNA, the concentration, yield and Cy3 uptake rate were calculated and the synthetic size (200 to 2000 nt were amplified) was measured. Six hundred ng of the labeled cRNA was fragmented at 60° C. and hybridized at 65° C. for 17 hours, and the array was washed and scanned.

A probe with the measured value reliable was extracted under the condition of either the control sample or the drug-added sample (one type), and the probe having an expression difference of 15 times or more was extracted as compared to the control sample.

(2) Results of Experiment

Tables 2 to 7 show mRNAs whose expression was increased by 15 times or more after treatment with the drug as compared to those before treatment, and their multiplication factor.

TABLE 2
UMBILICAL CORD-DERIVED MSCs

| | INCREASED | MULTIPLICATION |
|---|---|---|
| DRUG | GENE | FACTOR |
| TNFα | CSF2 | 51.92866 |
| IFNγ | SPARCL1 | 24.166359 |
| IL1α | CSF2 | 65.41428 |
| | CCL3 | 44.713257 |
| | MMP3 | 17.676903 |
| IL1β | CSF2 | 45.73727 |
| FGF15 | MMP7 | 20.78586 |

TABLE 3
ADSCs

| | INCREASED | MULTIPLICATION |
|---|---|---|
| DRUG | GENE | FACTOR |
| TNF-α | CSF3 | 457.4336 |
| | CSF2 | 210.81802 |
| | MMP9 | 36.619884 |
| | LIF | 28.586313 |
| | FGF13 | 26.435118 |
| | BMP2 | 24.754152 |
| | MMP3 | 16.642172 |
| TNF-β | MMP9 | 50.442356 |
| | CSF3 | 23.138222 |
| | FGF5 | 15.041612 |
| IFNβ | CSF3 | 81.64009 |
| | CSF2 | 81.4202 |
| | BTC | 37.966434 |
| | FGF20 | 33.667175 |
| IFNγ | FGF20 | 17.39512 |
| | MMP25 | 15.073852 |
| IL-1α | CSF2 | 3650.884 |
| | CSF3 | 3004.3464 |
| | MMP3 | 153.17429 |
| | MMP12 | 48.928066 |
| | LIF | 44.622696 |
| | NTN1 | 19.101036 |
| | HBEGF | 17.43808 |
| | MMP1 | 16.788137 |
| | MMP8 | 13.74857 |
| IL-1β | CSF3 | 3658.2717 |
| | CSF2 | 2945.6265 |
| | MMP3 | 163.10434 |
| | MMP12 | 77.35472 |
| | LIF | 44.191887 |
| | MMP8 | 20.121588 |
| | HBEGF | 17.731503 |
| | MMP1 | 17.499699 |
| | ADAMTS8 | 16.701633 |
| | FGF13 | 15.317384 |
| FGF15 | CSF3 | 1992.8231 |
| | CSF2 | 177.31819 |
| | MMP3 | 87.44734 |
| | MMP12 | 18.993986 |

TABLE 4
SYNOVIUM-DERIVED MSCs

| DRUG | INCREASED | MULTIPLICATION |
|---|---|---|
| TNFα | CSF3 | 481.9497 |
| | MMP3 | 184.5146 |
| | MMP1 | 118.73493 |
| | CSF2 | 93.68834 |
| | RSPO3 | 78.78901 |
| | RSPO3 | 54.598293 |
| | MM212 | 37.193733 |
| | ANGPTL1 | 29.710234 |
| | LIF | 19.802446 |
| TNF-β | CSF3 | 133.98564 |
| | MMP3 | 79.96354 |
| | MMP1 | 57.18347 |
| | RSPO3 | 29.265568 |
| | RSPO3 | 22.514166 |
| IFNβ | ANGPTL1 | 30.57255 |
| | FGF20 | 17.808767 |
| IFN-γ | MMP25 | 18.338886 |
| IL-1α | CSF3 | 10575.728 |
| | MMP3 | 1345.4216 |
| | MMP1 | 244.72272 |
| | MMP12 | 163.36778 |
| | CSF2 | 142.88773 |
| | MMP13 | 28.037321 |
| | MMP10 | 21.2812 |
| | RSPO3 | 18.53049 |
| IL1β | CSF3 | 8791.783 |
| | MMP3 | 935.04913 |
| | MMP12 | 181.83107 |
| | CSF2 | 129.32849 |
| | MMP1 | 107.597824 |
| | ADAMTS16 | 33.420284 |
| | GDF3 | 16.958546 |
| | MMP13 | 16.034931 |
| | IGF1 | 15.101902 |
| FGF15 | CSF3 | 1407.7273 |
| | MMP3 | 490.81107 |
| | MMP12 | 162.75064 |
| | MMP1 | 58.44772 |
| | IGF1 | 58.422787 |
| | BMP6 | 20.44636 |

TABLE 5
ADMPCs

| | INCREASED | MULTIPLICATION |
|---|---|---|
| DRUG | GENE | FACTOR |
| BMP-3 | GCG | 17.480957 |
| | NRTN | 28.843102 |
| BMP-4 | REG4 | 75.89187 |
| BMP-6 | HDGFL1 | 65.01833 |
| CCL-3 | EGF | 15.73962 |
| CCL-5 | MMP26 | 29.093975 |
| | MMP13 | 17.447323 |
| CCL-8 | BMP7 | 17.2787 |
| CCL-9 | BMP10 | 28.345194 |
| CCL-15 | BMP7 | 17.304893 |
| CCL-19 | FGF22 | 47.299706 |
| CCL-20 | EGF | 34.020695 |
| CCL-21 | BMP10 | 30.952303 |
| CCL-23 | ADIPOQ | 18.862783 |
| CCL-26 | FGF10 | 15.473124 |
| CCL-28 | NMU | 22.596947 |
| CNTF | ADAMTS20 | 44.9686 |
| | EGF | 20.999825 |
| CT-1 | FGF6 | 16.76011 |
| CXCL5 | ADAM22 | 15.880237 |
| CXCL10 | ADIPOQ | 23.21833 |
| | LTBP3 | 15.590775 |
| FGF15 | CSF3 | 78.26141 |
| | MMP3 | 21.577183 |
| GALECTIN 1 | IGF1 | 17.737694 |
| IFNβ | PTN | 34.09604 |
| | ANGPTL1 | 23.657429 |
| IFNγ | ADAMTSS | 18.498682 |
| | GLDN | 93.92384 |
| | PGF | 33.75676 |
| | FGF21 | 16.46351 |
| IGF-2 | IGFL4 | 18.903763 |
| IL-1α | CSF3 | 1266.8649 |
| | CSF2 | 241.81323 |
| | MMP8 | 90.97638 |
| | MMP3 | 82.76877 |
| | MMP12 | 62.105858 |
| | FGF13 | 61.590416 |
| | MMP1 | 30.74703 |
| | MMP13 | 21.096134 |
| IL-1β | CSF3 | 414.8679 |
| | CSF2 | 215.49258 |
| | MMP8 | 82.693 |
| | MMP3 | 81.932846 |
| | MMP12 | 80.88991 |
| | FGF13 | 53.733124 |
| | MMP1 | 29.282549 |
| | MMP13 | 19.679766 |
| IL-7 | BMP3 | 15.619323 |
| IL-8 | EGF | 166.93967 |
| | ADAMTS20 | 27.746012 |
| | MMP7 | 15.016879 |
| IL-11 | OOSP2 | 27.152683 |
| IL-17 | CSF3 | 47.33713 |
| IL-24 | FGF20 | 16.994074 |
| IL-26 | MMP26 | 25.349789 |
| IL-31 | ADAM21 | 16.247 |
| IL-35 | NPPC | 27.392384 |
| | EPO | 21.361319 |
| TNFα | CSF3 | 760.726 |
| | FGF13 | 519.2593 |
| | MMP8 | 322.53482 |
| | CSF2 | 84.63178 |
| | MMP3 | 42.06483 |
| | MMP1 | 41.600243 |
| | MMP13 | 33.378723 |
| | MMP10 | 33.15327 |
| | FGL2 | 30.280602 |
| | ADAM8 | 28.907818 |
| | MMP9 | 24.30117 |
| | RSPO3 | 22.690260 |
| TNFβ | FGF13 | 52.990948 |
| | CSF2 | 27.081493 |
| | CSF3 | 25.747252 |
| | MMP8 | 23.552876 |
| | MMP9 | 18.909662 |
| | MMP1 | 12.20611 |
| | MMP3 | 15.417225 |
| TRAIL | OOSP2 | 30.486296 |

TABLE 6
PLACENTA-DERIVED MSCs

| | INCREASED | MULTIPLICATION |
|---|---|---|
| DRUG | GENE | FACTOR |
| TNFα | CSF2 | 359.60034 |
| | MMP1 | 157.07306 |
| | FGF13 | 138.73257 |
| | CSF3 | 52.898098 |
| | MMP9 | 52.12447 |
| | RSPO3 | 36.97124 |
| | MMP12 | 28.326378 |
| | LIF | 20.185322 |
| TNFβ | CSF2 | 116.80491 |
| | MMP1 | 35.85062 |
| | RSPO3 | 33.528667 |
| | FGF13 | 30.268114 |
| IFNβ | ANGPTL1 | 67.8647 |
| IL1α | CSF3 | 4009.4556 |
| | CSF2 | 925.7604 |
| | MMP1 | 86.76511 |
| | MMP12 | 44.755383 |
| | EGF | 32.053967 |
| | MMP3 | 30.25197 |
| | MMP20 | 19.222319 |
| IL1β | CSF2 | 2408.85 |
| | CSF3 | 6519.517 |
| | EGF | 24.028 |
| | FGF13 | 18.65617 |
| | MMP1 | 143.90538 |
| | MMP12 | 87.97726 |
| | MMP3 | 50.21765 |
| FGF15 | CSF3 | 653.6217 |
| | CSF2 | 235.56853 |
| | MMP1 | 16.586615 |

TABLE 7
BONE MARROW-DERIVED MSCs

| | INCREASED | MULTIPLICATION |
|---|---|---|
| DRUG | GENE | FACTOR |
| TNFα | MMP1 | 62.074005 |
| | MMP3 | 39.413765 |
| | CSF2 | 34.723072 |
| | RSPO3 | 20.590866 |
| TNFβ | MMP1 | 16.974045 |
| IFNβ | BTC | 23.89296 |
| | DLL1 | 20.705479 |
| | PTN | 16.424856 |
| IFNγ | ANGPTL5 | 98.50543 |
| | RSPO3 | 17.248734 |
| IL1α | MMP3 | 545.55664 |
| | CSF2 | 248.18904 |
| | MMP12 | 81.264694 |
| | MMP1 | 20.65767 |
| | CSF3 | 17.709955 |
| IL1β | CSF2 | 544.1642 |
| | MMP3 | 482.9409 |
| | MMP12 | 55.837635 |
| | NTN1 | 34.482384 |
| | MMP1 | 28.635344 |
| | CSF3 | 17.319433 |
| FGF15 | MMP3 | 46.422943 |
| | MMP1 | 21.592436 |

In any of the experiments, it was confirmed that expression of polypeptides, growth factors and/or enzymes involved in tissue healing was enhanced in adherent cells derived from mesenchymal tissue treated with a physiologically active polypeptide. In many combinations of the physiologically active polypeptide and adherent cells derived from mesenchymal tissue, CSF2 and/or CSF3 tended to be expressed, in particular highly expressed. From these results, in the present invention, it has been found that a wide variety of physiologically active polypeptides and adherent cells derived from mesenchymal tissue can be used.

### INDUSTRIAL APPLICABILITY

The present invention is useful in the field of medicines for tissue healing and in the field of research of diseases requiring tissue healing.

1. A pharmaceutical composition for tissue healing, comprising adherent cells derived from mesenchymal tissue treated with a physiologically active polypeptide or lipopolysaccharide (LPS), and a pharmaceutically acceptable carrier.

2. The pharmaceutical composition according to claim 1, wherein the physiologically active polypeptide is one or more polypeptides selected from the group consisting of inflammatory cytokine, inflammatory cytokine-inducing polypeptide, growth factor, chemokine, hormone and interferon.

3. The pharmaceutical composition according to claim 1, wherein the physiologically active polypeptide is one or more polypeptides selected from the group consisting of interferon-β (IFN-β), interferon gamma (IFNγ), interleukin-1 alpha (IL-1α), interleukin-1 beta (IL-1β), interleukin-17A (IL-17A), tumor necrosis factor alpha (TNFα), tumor necrosis factor beta (TNFβ), type I interferon (INF-I), transforming growth factor β (TGFβ), epidermal growth factor (EGF) and fibroblast growth factor (FGF).

4. The pharmaceutical composition according to claim 1, wherein the adherent cells derived from mesenchymal tissue are mesenchymal tissue-derived stem cells (MSCs), adipose tissue-derived multilineage progenitor cells (ADMPCs), umbilical cord tissue-derived cells, placenta tissue-derived cells, or bone marrow tissue or synovium tissue-derived cells.

5. The pharmaceutical composition according to claim 1, wherein the tissue healing is tissue protection, repair of tissue/cell injury, promotion of proliferation of cells constituting a tissue, suppression of tissue inflammation, wound healing or reconstruction of tissue form.

6. The pharmaceutical composition according to claim 1, wherein the tissue healing is tissue healing in chronic phase disease.

7. The pharmaceutical composition according to claim 1, wherein the tissue is liver tissue or cardiac tissue.

8. A method for producing a pharmaceutical composition for tissue healing, comprising the steps of:
(a) treating adherent cells derived from mesenchymal tissue with a physiologically active polypeptide or LPS; and
(b) mixing the cells treated in step (a) with a pharmaceutically acceptable carrier.

9. The method according to claim 8, wherein the physiologically active polypeptide is one or more polypeptides selected from the group consisting of inflammatory cytokine, inflammatory cytokine-inducing polypeptide, growth factor, chemokine, hormone and interferon.

10. The method according to claim 8, wherein the physiologically active polypeptide is one or more polypeptides selected from the group consisting of interferon-β (IFN-β), interferon gamma (IFNγ), interleukin-1 alpha (IL-1α), interleukin-1 beta (IL-1β), interleukin-17A (IL-17A), tumor necrosis factor alpha (TNFα), tumor necrosis factor beta (TNFβ), type I interferon (INF-I), transforming growth factor β (TGFβ), epidermal growth factor (EGF) and fibroblast growth factor (FGF).

11. The method according to claim 8, wherein the adherent cells derived from mesenchymal tissue are mesenchymal tissue-derived stem cells (MSCs), adipose tissue-derived multilineage progenitor cells (ADMPCs), umbilical cord tissue-derived cells, placenta tissue-derived cells, or bone marrow tissue or synovium tissue-derived cells.

12. The method according to claim 8, wherein the tissue healing is tissue protection, repair of tissue/cell injury, promotion of proliferation of cells constituting a tissue, suppression of tissue inflammation, wound healing or reconstruction of tissue form.

13. The method according to claim 8, wherein the tissue healing is tissue healing in chronic phase disease.

14. The method according to claim 8, wherein the tissue is liver tissue or cardiac tissue.
